(19) United States
(12) Patent Application Publication
(10) Pub. No.: US 2025/0342931 A1
(43) Pub. Date: Nov. 6, 2025

(54) TAMPER-RESISTANT CONTAINER WITH UNIQUE IDENTITY AUTHENTICATION AND NETWORK-ENABLED SECURITY FEATURES

(71) Applicant: Impruvon, Inc., King George, VA (US)

(72) Inventors: Justin Michael Amoyal, Ashburn, VA (US); Michael Jared Mazzocco, King George, VA (US)

(73) Assignee: Impruvon, Inc., King George, VA (US)

(21) Appl. No.: 19/217,409

(22) Filed: May 23, 2025

Related U.S. Application Data
(63) Continuation of application No. 17/496,538, filed on Oct. 7, 2021, now Pat. No. 12,315,612.
Application No. 17/496,538 is a continuation-in-part of application No. 16/923,641, filed on Jul. 8, 2020, now Pat. No. 11,166,879.
(60) Provisional application No. 62/871,426, filed on Jul. 8, 2019.

(51) Int. Cl. G16H 20/13; G06F 21/31

(52) U.S. Cl. CPC G16H 20/13; G06F 21/31

(57) ABSTRACT

A medication system for dispensing prescription medications. The medication system has a software application adapted to provide access to a plurality of modular containers secured by a locking mechanism. The software application is adapted to collect data regarding individuals receiving care to automate documentation for regulatory compliance. The software application can also provide guided medication adherence instructions.

### CROSS-REFERENCE TO RELATED APPLICATIONS

The present application is a continuation of U.S. patent application Ser. No. 17/496,538, filed Oct. 7, 2021, which in turn is a continuation in part of U.S. patent application Ser. No. 16/923,641, filed July 8, 2020, which in turn claims the benefit of U.S. Provisional Application No. 62/871,426, filed Jul. 8, 2019, the contents of all the aforementioned are hereby incorporated by reference in their entirety.

### STATEMENT REGARDING FEDERALLY SPONSORED RESEARCH OR DEVELOPMENT

The invention described herein was made without federal funding.

### BACKGROUND OF THE INVENTION

The present invention relates to the technical fields of medicine, pharmacy and telehealth. More particularly, the embodiments of the present invention include improved means for the secure dispensing of medications leveraging a combination of specialized medication dispensers with modern software applications and network communications technologies. The dispensers described herein are tamper resistant, may require a unique identification to authenticate the identity of the user and enable mobile locking, unlocking and alarming that assimilates with text messaging and smartphone capabilities via software applications and application programming interfaces (API). The tamper-resistant containers may also be configured with audio, visual or vibratory alarms to alert users of unauthorized attempts to access the contents, as well as displays used as rewards for adherence to prescription dosing regimens. The tamper-resistant containers will be dynamic in the sense of inputs to the device, and is thus capable of accepting unit dose (single dose and multi-dose) medication packages. Designs will be dynamic to support loading, guiding, and dispensing blister packs, pill packets, pill bottles, and other medication packaging systems filled by pharmacies.

Existing conventional medication dispensers lack mobile locking, unlocking and alarming capabilities, unit-dose packaging integration capability, and are not accompanied with unique user identity authentication. Generally speaking, the patient or other user must open a childproof pill lid, for example, to gain access to the medication inside the bottle or case. With conventional pill bottles, the patient's medication is vulnerable to being stolen, moved, tampered with and not completely secured, and provides no mechanism to alert the patient or a patient custodian if tampering occurs. It is thus an object of the invention of the present disclosure to provide a secure, tamper-resistant container that leverages cloud computing and software application technologies to prevent unauthorized access to medications and alert users when attempts at such unauthorized access are made, as well as provide a mobile means of locking and unlocking containers of medicine. It is a further object of the invention to provide an interactive mobile software application for rewarding patients for prescription compliance with games, avatars and the like visible on a graphical user interface (GUI) to encourage patients suffering from autism or other similar disabilities, for example, affecting compliance to take the appropriate doses of necessary medications timely.

It is yet another object of the present invention to provide institutions such as nursing homes and the like with a system designed to improve patient adherence to single or multiple prescriptions and enable remote refilling as needed. It is another object of the invention to enable such institutions to receive updates from doctors' offices and pharmacies regarding changes to medication regimens for their residents. These and other benefits of the invention described herein will be evident to those of ordinary skill in the art in the applicable technical fields.

### SUMMARY OF THE INVENTION

The present invention comprises tamper-resistant containers of resilient material that may include network enabled security features for improved medication security and adherence. In one aspect, containers according to the present invention are fabricated from durable plastic material and are configured to dispense multiple single dose packs or units contained in multi-unit blister packs. The latter may comprise punches for automated dispensing the pills contained in each blister pack. A container according to the present disclosure may also be configured with a power source, such as a rechargeable battery with a universal serial bus (USB) port, that powers a unique user identity authentication sensor, such as but not limited to a fingerprint biometric scanner, in combination with a vibration sensor to detect container movement. In certain embodiments, biometric authentication capabilities may be provided via a mobile app as described herein, as will be understood by one of ordinary skill in the art. The user identity authentication and vibration sensors may communicate via Bluetooth transceiver to a smartphone or via an available wireless network to a cloud server comprising software program instructions which when executed by a processor cause the processor to process the data inputs for authentication or transmit alerts to untimely or unauthorized access to a smartphone configured to receive messages on a GUI or via short message service (SMS) text in communication with the cloud server via wireless network.

For example, and not by way of limitation, a smartphone may receive text messages and audio, visual or vibratory alerts, and may be configured to enable authenticated, remote locking or unlocking of an associated container. A system of the present disclosure may also enable updates and reminders to be sent to a smartphone regarding dosing times, the need for refills, and changes to dosing regimens. These features provide the following benefits: (1) instantly alerting of parents, friends or loved ones of unauthorized users attempting to steal pills; (2) simplification of the outdated methods of opening pill bottles and cases through access only upon authentication, such as by fingerprint reading or another suitable authentication means; and (3) eliminating unauthorized users from breaking into or cutting open pill bottles or cases to steal pills by using tough, durable manufacturing material.

The secure dispensers of the present disclosure may integrate with smartphones via an API or native mobile application installed thereon or responsive web application accessed thereon, which enables text messaging to provide users with an easy to use link for live updates and locking and unlocking of the system. In addition, users benefit from modern mobile application capabilities that provide real-time awareness and access to medication data for individuals, their guardians, or healthcare providers. An application according to the present disclosure may also provide for data collection and logging to gain insights into historical performance of the dispensers and adherence to prescriptions. Gamified education and training is also enabled to assist patients with various disabilities with medication compliance. The mobile application functionalities may also be enabled in the form of a web application accessible via an Internet website provided by the applicant or third party. These and other benefits of the invention of the present invention will be apparent to one of ordinary skill in the art in view of the disclosure that follows.

An aspect of the present disclosure is a medication system. The medication system comprising: a plurality of modular containers and a locking mechanism configured to secure medication within the plurality of modular containers, wherein at least one of the plurality of modular containers can be accessed via a software application adapted to provide access to plurality of modular containers; and wherein the software application is further adapted to provide an alert to take medication, receive confirmation that the medication was taken, and automatically provide a reward upon confirmation the medication was taken.

Another aspect of the disclosure is a medication system. The medication system comprising: a software application adapted to provide access to a plurality of modular containers, wherein the plurality of modular containers are secured by a locking mechanism, configured to secure medication within the plurality of modular containers, wherein the software application is further adapted to provide an alert to take medication, receive confirmation that the medication was taken, and automatically provide a reward upon confirmation the medication was taken.

Still yet another aspect of the disclosure is a medication system. The medication system comprising: a plurality of modular containers; a software application adapted to provide access to the plurality of modular containers, wherein the plurality of modular containers are secured by a locking mechanism configured to secure medication within the plurality of modular containers, wherein the software application is further adapted to provide an alert to take medication, receive confirmation that the medication was taken, and provide access to compliance logs.

### BRIEF DESCRIPTION OF THE DRAWINGS

FIG. 1 is an exploded view of an exemplary embodiment of a medication dispenser according to the present disclosure.

FIG. 2 illustrates multiple connected units as illustrated in FIG. 1 with lids closed.

FIG. 3A is an upper perspective view of a blister pack dispenser according to an embodiment of the present disclosure.

FIG. 3B is an upper perspective view of a container as illustrated in FIG. 3A, with blister packs loaded.

FIG. 4 illustrates a connected series of closed containers according to the present disclosure, including avatar displays on some units and a fingerprint scanner for secure access.

FIG. 5 illustrates a secure blister pack dispenser according to an embodiment of the present disclosure, configured for automated dispensing of medications.

FIG. 6 illustrates an exemplary system architecture for enabling certain container features described herein and the claimed methods.

FIGS. 7A-7C illustrate screenshots from a web application as described herein.

FIGS. 8A-8C illustrate a first set of screenshots from a mobile application as described herein.

FIGS. 9A-9C illustrate a second set of screenshots from a mobile application as described herein.

### DETAILED DESCRIPTION OF THE INVENTION

The invention of the present disclosure comprises a durable, secure, medication dispenser, which may be used in connection with a provided software application which can run on a web server or be downloaded to a mobile device such as a smartphone or tablet and be utilized by authenticated users to dispense and refill medications, or be used to provide rewards to individual patients for medication compliance. It is an object of the present invention to ease access to medications and prescription compliance, and to reward compliance with video games and graphics viewable on a smartphone, for example. It is another object of the invention to permit only authorized access to medications contained in the dispenser, alert users of a system as described herein to unauthorized or unscheduled attempts to dispense medications therefrom, and improve compliance with prescription dosing regimens, particularly in cases where the individuals to whom the medications are prescribed have some form of difficulty adhering to prescription schedules.

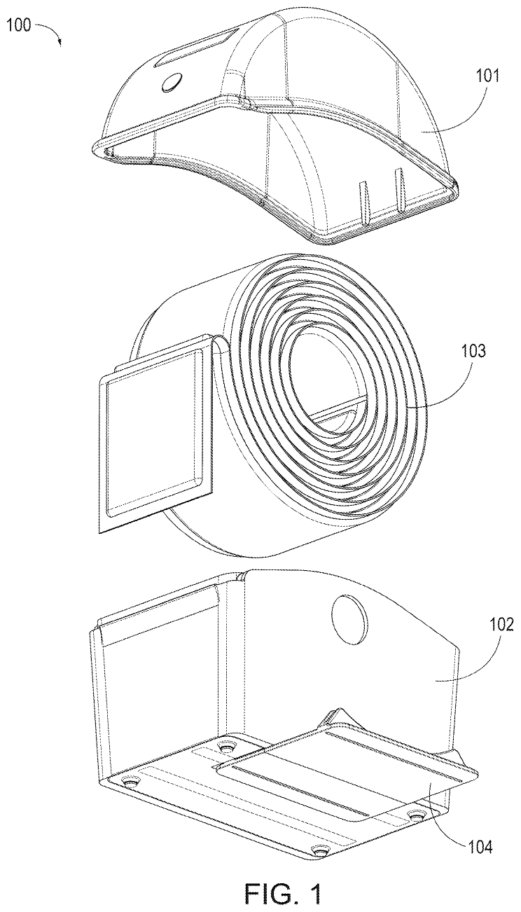

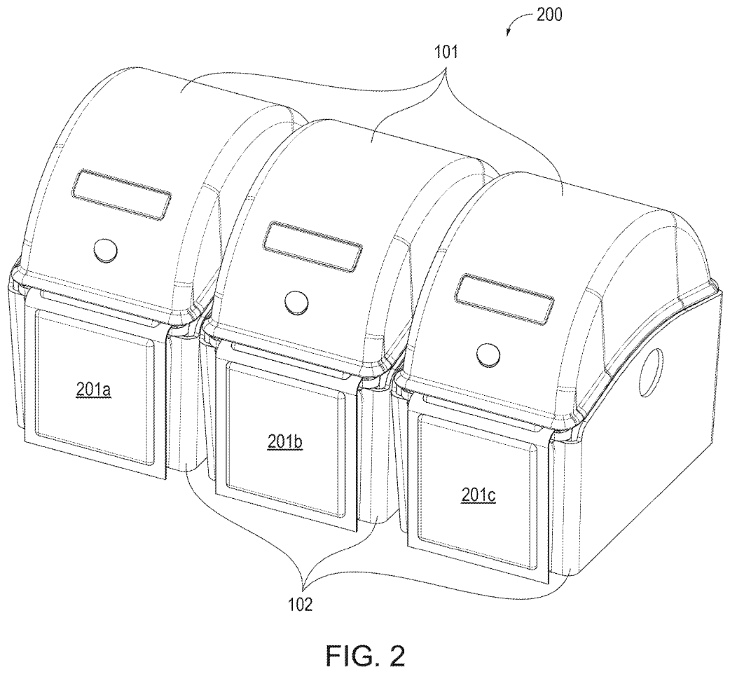

The invention of the present disclosure will be best understood with reference to the accompanying drawings. FIG. 1, for example, illustrates an embodiment of a container according to the present invention configured for dispensing of single packets, each containing one or more medications, of a connected series of packets. This illustrative embodiment comprises a lid that fits securely over a container, the lid and container being fabricated of durable material and of suitable size to contain a roll of individual medication packets that may be dispensed one at a time. Each unit as shown in FIG. 1 may be equipped with a base connector to enable connection of a series of units, as illustrated in FIG. 2, each containing a roll of a different medication, for example. In the case of institutional use, each unit may be loaded with medications for a different patient and configured with network-enabled security features for automated dispensing in response to instructions provided by a user of a software application as described herein. Alternatively, an individual may self-dispense in response to an alert to take a dose of medication, and dispensing may trigger a reward in the form of a game or graphic display visible on a smartphone of the individual taking the medication.

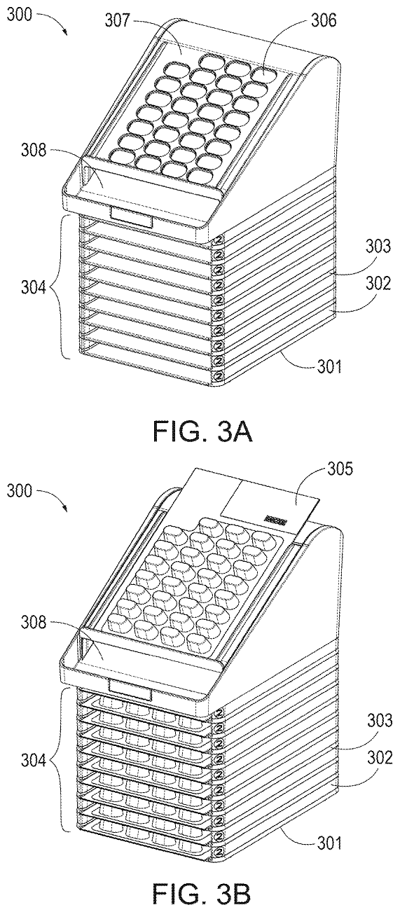

An exemplary dispenser according to the present disclosure may be configured to accommodate several blister packs containing several doses of medication (e.g., pills or capsules), as illustrated in FIGS. 3A (empty) and 3B (loaded). As shown in FIGS. 3A and 3B, a base tray and a plurality of spacers and upper trays may be connected to create a magazine containing multiple blister packs for manual distribution by pushing each pill or capsule through an opening in a top tray configured with a plurality of openings of suitable size and shape to accommodate a corresponding blister pack. A receiving tray may be provided to receive a dispensed medication when pushed through an opening. As is the case with the design illustrated in FIG. 1, base connectors may be provided so multiple dispensing units according to FIGS. 3A and 3B may be connected, each used to house individual prescription medications for individuals requiring multiple medications, or in the case of institutional use, individual medications for different patients, in order to prevent confusing the prescriptions for one another.

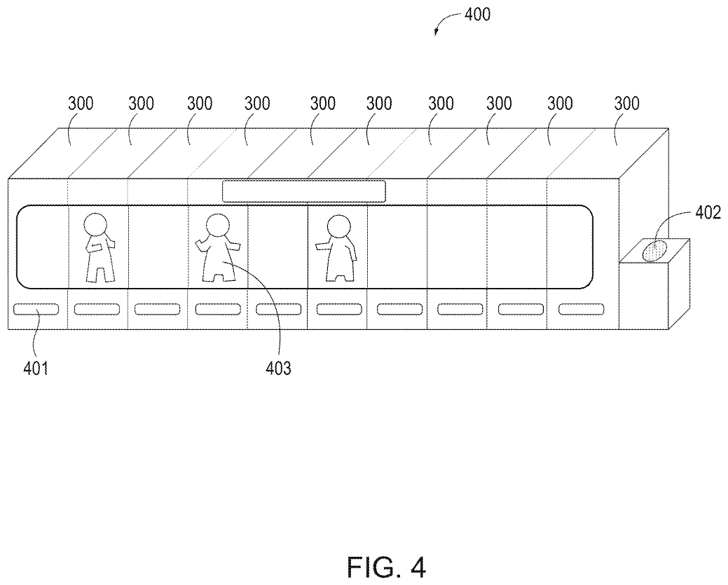

Each unit according to FIGS. 3A and 3B may be secured by providing a cover of durable material that fits over the entire unit, as illustrated in FIG. 4. Continuing with FIG. 4, an authentication means such as a fingerprint scanner may be provided, along with LED lighting and screens on which graphics may be displayed as rewards for timely dispensing of medications in response to instructions provided using a connected mobile application after authentication. Each cover shown in FIG. 4 is configured with an opening though which medications may be dispensed by gravity after authorization. For prescription compliance training for individuals suffering from difficulties in determining which medications to take and when, adherence may be gamified and rewards such as the display of avatars as illustrated in FIG. 4 may be provided as an additional feature of the present invention.

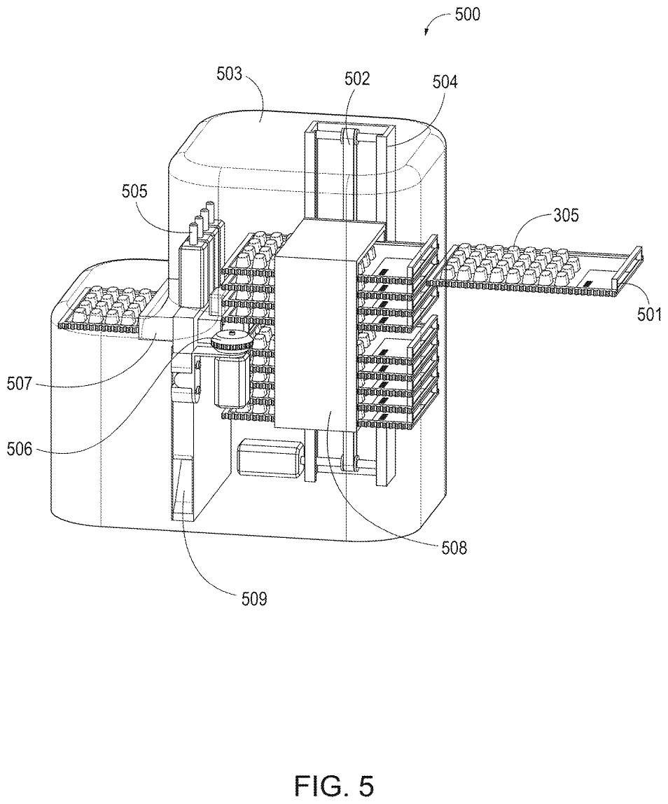

Certain embodiments of a container according to the present disclosure may be configured with various mechanical parts to enable automated dispensing of dosing regimens comprising a plurality of blister packs as shown in FIG. 5. In this illustrative embodiment, a secure housing contains a magazine, in turn containing a plurality trays and spacers, similar to those described with reference to FIGS. 3A and 3B, is held within a sliding bracket configured with a guide pin (not shown) secured within a guide channel for mechanically raising and lowering in response to instructions provided by a connected software application to dispense appropriate medications. The unit illustrated in FIG. 5 is configured with a row of punches corresponding to the locations of blisters within each row and column within a blister pack. The unit of FIG. 5 is configured with a conveyor that moves each blister pack forward toward the punches in order to line the medications up with the punches as needed, then each punch can be selected for activation and push the medication contained in the assigned blister out of the pack and into a chute where it is gravity-dispensed.

In certain embodiments, a plurality of punches may be provided to punch the required blister for the designated time and date of a dose of a medication contained therein. Magazine cartridge options may include but are not limited to 5, 10, 15 and 20 single blister pack magazine cartridges in vertical lift. Individual blister pack cartridges may include horizontal gear motion controls to place the medication in the blister pack in the correct position for punching. Gravity dispensing of individual medication units from the punched blister pack to a reservoir below may be employed. A spring loaded gear pass through control may also be provided. Radio frequency identification (RFID) sensors, barcodes or the like may be employed in order to collect medication data from its packaging, for example, or alternative means may be used as will be familiar to one of ordinary skill in the art. Individual units may be provided in series to better serve the needs of those requiring multiple medications.

A web or mobile application according to the present disclosure may be configured for use either by the individual to whom medications have been prescribed, or by caregivers, including parents, guardians, or institutional caregivers such as nursing staff and doctors. The user interfaces for each type of user are differentiated for customized, appropriate user experiences.

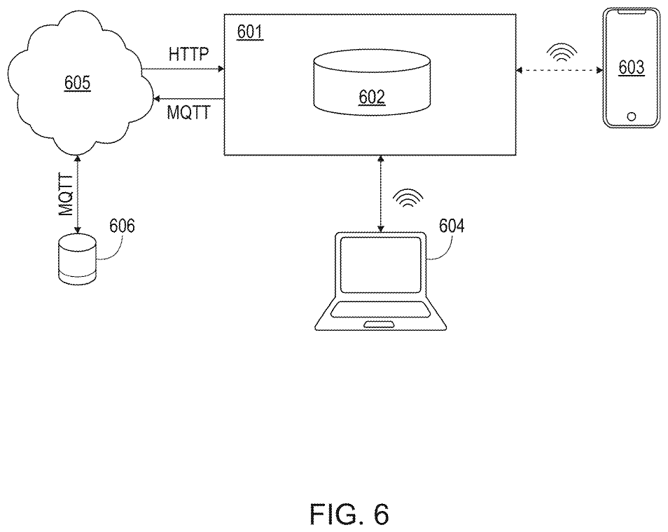

The invention of the present disclosure is supported by a backend system comprising components such as those illustrated in FIG. 6. In this exemplary embodiment, a web server 601 in network communication with a cloud server 605 via Hypertext Transfer Protocol (HTTP) (incoming) and Message Queuing Telemetry Transport (MQTT) protocol (outgoing), the cloud server 605 being in wireless communication with a tamper-resistant container according to the present disclosure via MQTT, supports system functionality. A smartphone 603, for example, configured with an API for interaction with a computer software application tangibly stored on a non-transitory computer readable medium, support functionality of a mobile application as described herein. For mobile applications, a cloud server 605 may support the running of the application. Data inputs from a smartphone 603 (or computer via website) are processed according to the instructions contained in the software application by a microprocessor in network communication with a cloud server 605, for example. Processing includes but is not limited to user authentication, locking or unlocking of a medication dispenser, or alerting or messaging the smartphone 603. The system is designed to anonymize all data traffic to and from a container as described herein, thereby maintaining the data privacy of individual users of a system comprising a container as described herein. Data collected and stored within a system of the present disclosure may be stored on a database 602 and related to the utilization of a container as described herein to enable analytics for users. Updates can be pushed out by an administrator to the web server 601 using any suitable computing means 604.

Users of a system as described herein determine who should have access to the system and set permissions according to their role as user, custodian, physician, etc. Users may input information on medications, dosages, dosage regimens and so forth to better manage adherence to prescription requirements. In preferred embodiments, a container according to the present invention may hold up to four weeks of medication schedule to be dispensed up to four times per day, although this is by way of example and not limitation. A system of the present disclosure may be configured to interface with pharmacies through telehealth programs to enable seamless refilling and delivering of medications prior to running out.

In certain embodiments, a system of the present invention also may provide guided or interactive medication adherence instructions for custodians or individuals taking medications. These may be communicated via audiovisual means over a computing means such as a smartphone. It is an object of the present invention to teach better medication adherence to individuals who suffer from intellectual developmental disabilities (IDD) such as Autism Spectrum Disorder (ASD) or attention deficit disorder who may have difficulty focusing on proper adherence, individuals undergoing recovery from addiction, veterans battling post-traumatic stress disorder (PTSD), the geriatric population with difficult to manage medication regimens and/or individuals looking to improve medication management practices in their homes.

Specific embodiments of the present invention comprise a tamper resistant pill dispenser with unique identity authentication and mobile locking, unlocking and alarming capability, integrated into a system comprising a computer software application accessible via a smartphone or physically located on the dispenser. Future enhancements may be made based on system testing along with user input and feedback.

It is an object of the present invention to provide alerts, enable remote locking and unlocking, and communicate updates and reminders related to medication schedules, refill needs and the like. It is also an object of the invention of the present disclosure to enable the authenticated user to access medications contained inside the container only following authentication such as by fingerprint scanning. While a medication dispenser according to the present disclosure may be made of stainless steel, aluminum, or of any other sufficiently rigid and strong material such as high-strength plastic, metal, composites and the like, the various components of the dispenser can be made of different materials. A dispenser may also be configured with a Bluetooth transceiver for short wave transmission of data to a smartphone or the like in wireless network communication with a cloud server in further communication with a web server comprising a software application tangibly stored thereon which supplies instructions which when executed by a processor cause the processor to process data from the dispenser, such as but not limited to vibration, motion or authentication data.

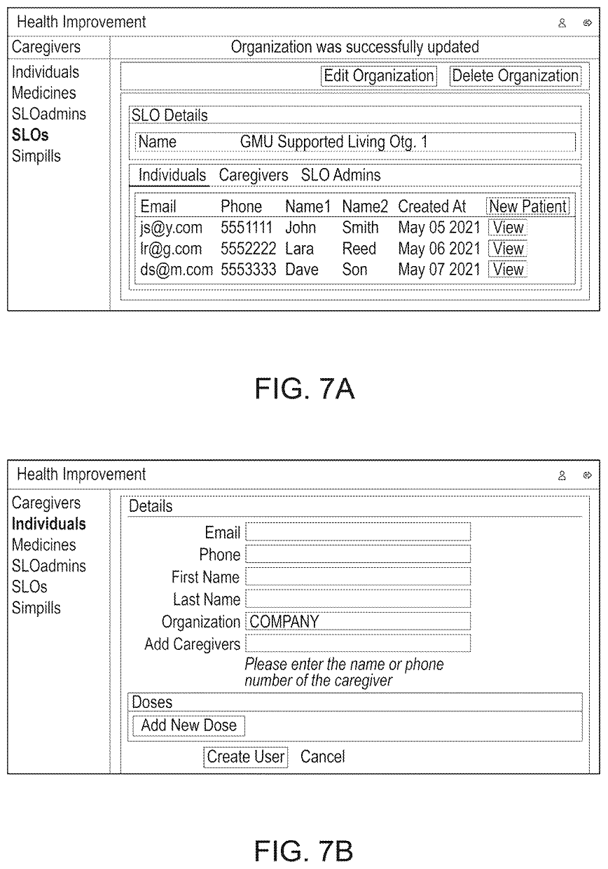

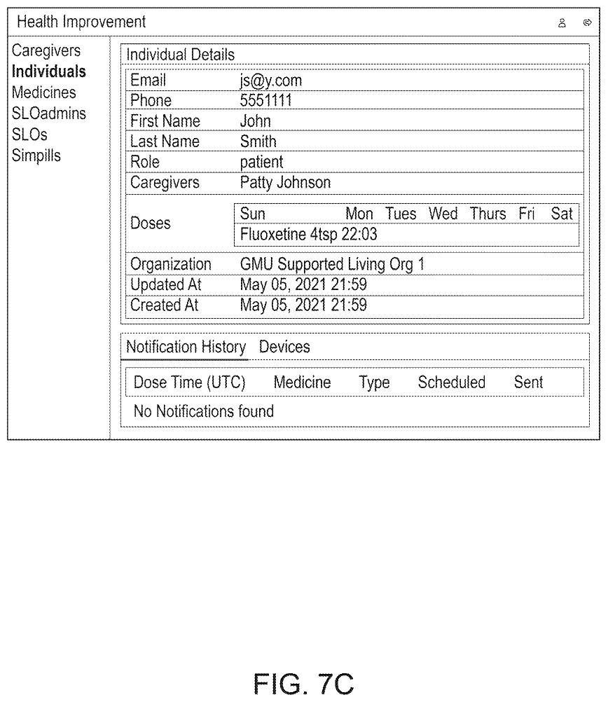

As explained briefly throughout the preceding disclosure, a system according to the present disclosure may be accessible in the form of a web application available over the Internet on a website, or a mobile application downloadable to a compatible device. As shown in FIGS. 7A-7C, a web application is designed to provide a top-down view for administrators to view all individual associated patients benefitting from the system. For example, a long-term care agency administrator may include all caregivers and other staff associated with medication management at his or her institution for access, as illustrated in FIG. 7A. Data visualizations and data analytics may be included on a dynamic dashboard providing a single site for key performance indicators (KPIs) to be displayed to web application users. Users may create various profiles according to known practices in the art, with administrators having the ability to create, edit and delete users within their organizations and all necessary fields for use of the system, including personal information, prescription information, and so on. Where the user of the system is an individual, self-administration may be desired.

As shown in FIG. 7B, an administrator may populate all the necessary fields appropriate for medication management for a given individual within an institution, for example. Alternatively, integrations are developed with existing software tools run within the pharmacy environment to automate medication regimen inputs via APIs. For example, a pharmacy supporting an Assisted Living Organization that is using the platform these claims refer to could push all medication regimen data for the Assisted Living Organization to the cloud-based database to then be reflected in the mobile and web applications. Turning to FIG. 7C, fields so populated are illustrated. A system of the present disclosure provides an interface to input medication regimen data. Users can input full medication regimens for individuals, such as schedule (day, time, and frequency per day of dosing), medication type, dose amount, expiration date, refill date, etc. Pharmacy software integrations may be used to automate the otherwise manual entry of this data. A system according to the present disclosure may enable a feedback loop between pharmacies and long-term care agencies for real-time changes, such as when a medication change occurs (the pharmacy can update the regimen and update the system entries). In preferred embodiments, a web application of the present disclosure will also allow for data export based on different views, such as, for example, exporting a medication compliance history to share with medical professionals using compatible file types.

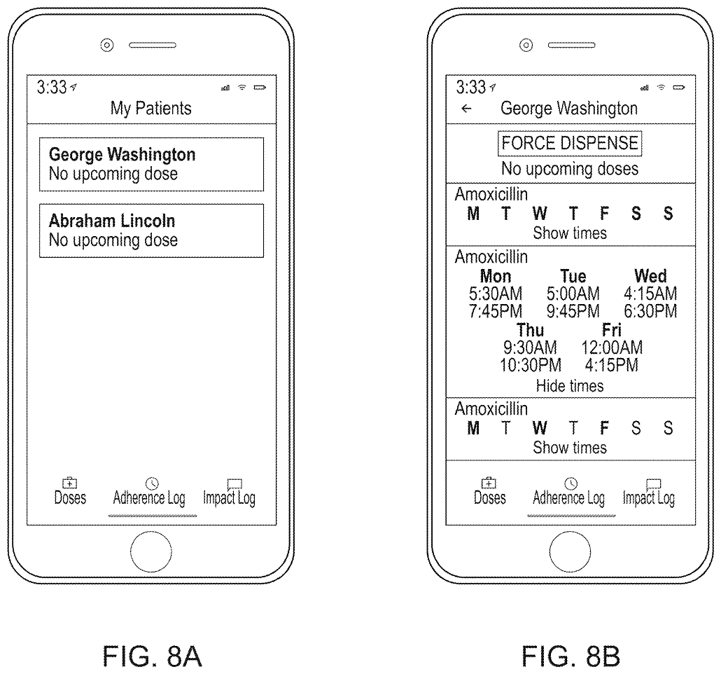

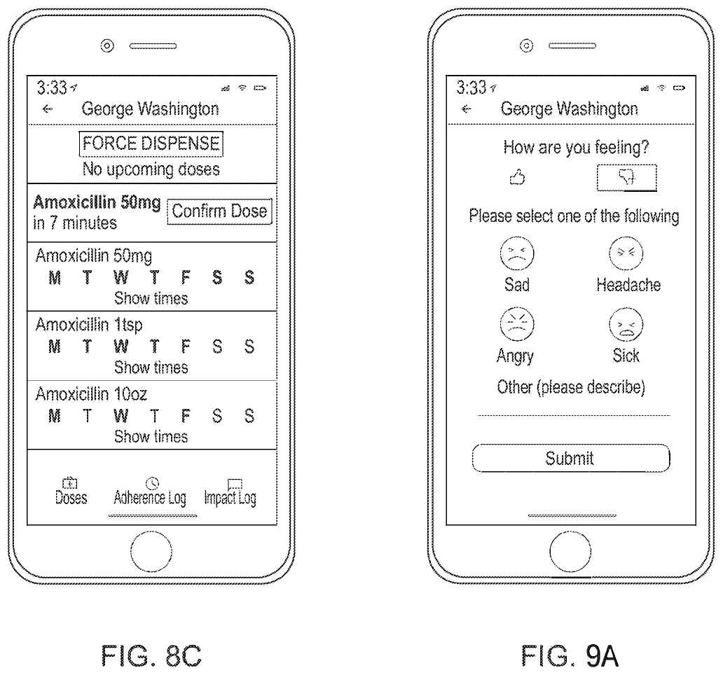

Turning now to a mobile application as contemplated herein, the app is designed to provide real-time awareness and access to medication data to individuals and their supporting personnel. Exemplary mobile screen views are illustrated in FIGS. 8A-8C. FIG. 8A names the patients associated with an administrator following secure login. In FIG. 8B, after selecting a patient that patient's full medication schedule becomes visible. From there, the administrator, caregiver, or individual can force dispense the confirmed dosage and ensure it is correct as shown in FIG. 8C depending on the preferences set per user.

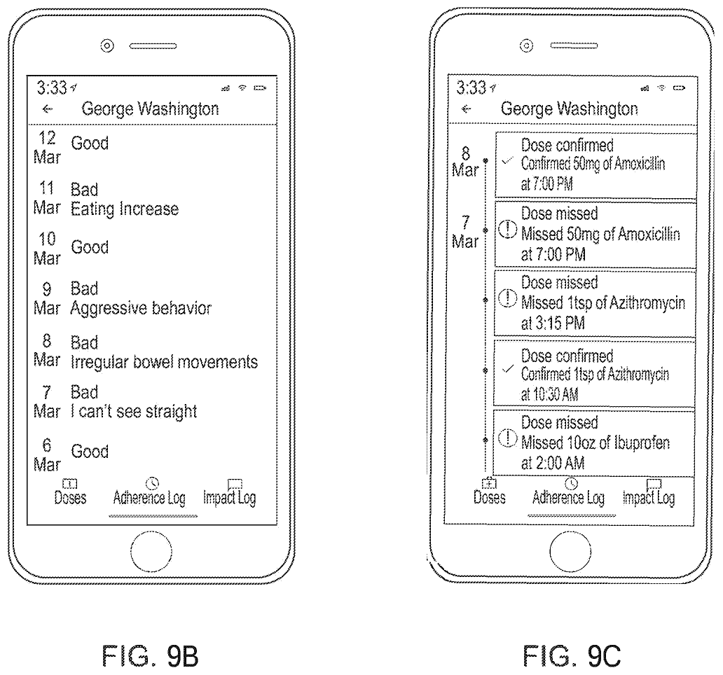

Reminders are provided when medications are due to be taken or administered, as well as when they expire or when refills are due. Alerts may be pushed to a user device if doses are missed or dispensed late, or if potential threats are detected, such as theft or overdose attempts. Data collection and logging for insights into historical performance may also be featured. For example, medication impact logging functionality may push a notification to a user asking how they are feeling then provide custom interfaces familiar to users where they can input data to describe how they are feeling and uncover reactions such as potential medication side effects, as illustrated in FIGS. 9A-9C.

Full historical logs of medication compliance for individuals are enabled by a system according to the present disclosure. A caregiver may be able to view logs for all associated individuals. Full historical logs of medication impacts driven by input taken from individuals as well as caregivers is possible.

A system of the present disclosure also provides gamified education and training for individuals having difficulty with medication adherence. Routine, repetition, and rewards-based experiences for individuals and caregivers are used to motivate users to comply with medications, understand the impacts of complying, and drive independence. Upon setup, users may be taken through a set of interactive surveys to understand their interests and behaviors, to be later introduced in the user experience. For example, a user may select types of games that he or she enjoys, and may unlock these types of games as they become more independent and advance through training modules. Using yet another example, a user may select types of characters they enjoy viewing, and an avatar may be generated for that individual to guide through training and education modules.

Preferred embodiments of mobile applications will allow users (depending on authorization settings) to interact with their devices. Examples of interactions include dispensing medications (e.g., user pressing a button in the app that makes the device dispense the correct dose at that time). Once medications are loaded, accessed, and dispensed from the device these steps in the process will be made relevant in the mobile app interface to provide the users with various insights over time. Regulatory automation may also be enabled by a system as described herein, with on object of automating the three-way check currently required at long-term care agencies. Rather than comparing a scanned copy of physical form of identification, for example, methods employed by the mobile app may include facial recognition to validate the identity of an individual. Rather than comparing the label on a doctor's order to the label on a medication, integrated pharmacy software may validate using the code which exists on the medication packaging currently stored in the device. Rather than remembering to look up medication administration instructions and preferences form a multitude of physical forms, a reminder may be sent to the medication administrator (caregiver) or the individual depending on permissions with a description.

The advantages of the systems and methods described herein include, without limitation, significant reduction in the ability to tamper with or break into the container and steal controlled substances or illegally access prescription medication, automatic notification to the owner if a break-in attempt occurs, simplicity of access for the content owner, and accessibility and data tracking via a computer software application, preferably accessible from a mobile computing device. Future enhancements over the current state of the art include, but are not limited to the use of a fingerprint scanner, for example, which may already be accessible by mobile phones and the like; integration of video, audio, camera and global positioning system (GPS) functions; automatic transmission of reminders when to take medication and warnings on side effects; and integration with Internet of Things (IoT) type devices; live results from wearable health monitors and sensors; alerts of drug interactions and lockout periods based on instructions for administration; and interfaces with available pharmaceutical prescription information, dispensing guidelines and directions of when to take the medication. The final shape, size, weight and dimensions of the invention may be customized through developmental, operational and user testing.

While the foregoing written description of the invention enables one of ordinary skill to make and use what is considered presently to be the best mode thereof, those of ordinary skill in the art will understand and appreciate the existence of variations, combinations, and equivalents of the specific embodiments, methods, and examples described herein. The invention should therefore not be considered limited according to the illustrative embodiments described herein, but by all embodiments and methods within the scope and spirit of the invention as claimed.

What is claimed:

1. A medication system comprising:
a plurality of modular containers and a locking mechanism configured to secure medication within the plurality of modular containers, wherein at least one of the plurality of modular containers can be accessed via a software application adapted to provide access to plurality of modular containers; and
wherein the software application is further adapted to provide an alert to take medication, receive confirmation that the medication was taken, and automatically provide a reward upon confirmation the medication was taken.

2. The medication system of claim 1, wherein at least one of the plurality of modular containers can be accessed using biometric data.

3. The medication system of claim 1, wherein the software application is further adapted to provide notification of unauthorized attempts to access at least one of the plurality of modular containers.

4. The medication system of claim 1, wherein the software application further provides access to compliance logs.

5. The medication system of claim 1, wherein the software application is adapted to provide guided medication adherence instructions.

6. The medication system of claim 1, wherein the software application is adapted to collect data regarding individuals receiving care to automate documentation for regulatory compliance.

7. The medication of system of claim 1, wherein the software application is adapted to receive live results from a wearable health monitor.

8. A medication system comprising:
a software application adapted to provide access to a plurality of modular containers, wherein the plurality of modular containers are secured by a locking mechanism, configured to secure medication within the plurality of modular containers,
wherein the software application is further adapted to provide an alert to take medication, receive confirmation that the medication was taken, and automatically provide a reward upon confirmation the medication was taken.

9. The medication system of claim 8, wherein at least one of the plurality of modular containers can be accessed using biometric data.

10. The medication system of claim 8, wherein the software application is further adapted to provide notification of unauthorized attempts to access at least one of the plurality of modular containers.

11. The medication system of claim 8, wherein the software application further provides access to compliance logs.

12. The medication system of claim 8, wherein the software application is adapted to provide guided medication adherence instructions.

13. The medication system of claim 8, wherein the software application is adapted to collect data regarding individuals receiving care to automate documentation for regulatory compliance.

14. The medication of system of claim 8, wherein the software application is adapted to receive live results from a wearable health monitor.

15. A medication system comprising:
a plurality of modular containers;
a software application adapted to provide access to the plurality of modular containers, wherein the plurality of modular containers are secured by a locking mechanism configured to secure medication within the plurality of modular containers,
wherein the software application is further adapted to provide an alert to take medication, receive confirmation that the medication was taken, and provide access to compliance logs.

16. The medication system of claim 15, wherein the software application is further adapted to automatically provide a reward upon confirmation the medication was taken.

17. The medication system of claim 15, wherein at least one of the plurality of modular containers can be accessed using biometric data.

18. The medication system of claim 15, wherein the software application is further adapted to collect data regarding individuals receiving care to automate documentation for regulatory compliance.

19. The medication system of claim 1, wherein the software application is adapted to provide guided medication adherence instructions.

20. The medication of system of claim 1, wherein the software application is adapted to receive live results from a wearable health monitor.
